(19) United States
(12) Patent Application Publication
(10) Pub. No.: US 2023/0190497 A1
(43) Pub. Date: Jun. 22, 2023

(54) ORTHOPEDIC DEVICE

(71) Applicant: OTTOBOCK SE & CO. KGAA, Duderstadt (DE)

(72) Inventors: Jens NÖRTHEMANN, Duderstadt (DE); André SCHMERGLATT, Landolfshausen (DE)

(21) Appl. No.: 18/005,214

(22) PCT Filed: Jul. 26, 2021
(86) PCT No.: PCT/EP2021/070829

§ 371 Date: Jan. 12, 2023

(30) Foreign Application Priority Data
Jul. 31, 2020 (DE) 10 2020 120 304.7

(51) Int. Cl. A61F 2/74

(52) U.S. Cl. CPC A61F 2/74; A61F 2002/5086

(57) ABSTRACT

The invention relates to an orthopedic device comprising at least one joint and a hydraulic system with at least one valve. The hydraulic system is filled with a hydraulic fluid and the hydraulic fluid contains a silicone oil (2) and an additional oil (4) which has at least one ester group.

The invention relates to an orthopedic device with at least one joint and a hydraulic system with at least one valve, the hydraulic system being filled with a hydraulic fluid. The invention also relates to a method for filling a hydraulic system for such an orthopedic device.

Orthopedic devices are known in particular from the prior art in the form of prostheses and orthoses, but also in the form of other supportive devices. This relates, for example, to exoskeletons and other devices that support the user of the device, for example, during strenuous work, for example above the head, so that the strenuous work can be performed for longer. Orthopedic devices according to the preamble feature at least one joint with a hydraulic system. For example, it may include a hydraulic damper. In many embodiments, the hydraulic system has multiple chambers that are fluidically connected to each other by a channel, duct or another method. For example, if the joint is now moved, the hydraulic fluid is conveyed from the one chamber into another chamber. Depending on the diameter of the fluidic connection between the two chambers in question, this results in a flow resistance against the movement, i.e. a damping. According to the preamble, this hydraulic system includes at least one valve that is used, for example, to vary the specified flow resistance. The valve can be open or closed or partially open and thus adjust the diameter available to the flow, for example the opening width, of the fluidic connection between the two chambers concerned.

Orthopedic devices according to the preamble consequently feature a hydraulic system with moving parts. The hydraulic fluid therefore has to exhibit different properties and perform different tasks. On the one hand, for example, the viscosity of the hydraulic fluid should vary as little as possible upon a change in temperature. Whether the orthopedic device is used, for example, in winter in temperatures below the freezing point of water or, for example, in 30° C. in summer, should have as little influence as possible on the viscosity of the hydraulic fluid. The viscosity is decisive for the flow properties of the hydraulic fluid. The higher the viscosity, the thicker and less flowable the hydraulic fluid. Of course, the flowability has a significant impact on how quickly or easily the hydraulic fluid is able to flow through the hydraulic system and particularly through the at least one valve. It therefore also influences flow resistance and thus damping. Consequently, a hydraulic fluid whose viscosity varies greatly depending on temperature would result in the damping resistance applied by the hydraulic system at a given valve position becoming temperature dependent, especially in the case of laminar throttling. This is not desirable and would negatively impact the level of comfort of the orthopedic device, or even mean a loss of stability of the orthopedic device.

At the same time, a hydraulic fluid should lubricate the moving parts that come into contact with the hydraulic fluid in order to minimize the resistance to movement, i.e. friction, of the moving parts against each other.

The use of various oils as hydraulic fluid is known from the prior art. For example, single-component and multi-component silicone oils are used, the viscosities of which exhibit a comparatively low dependence on temperature. In this case, multicomponent silicone oils are mixtures of multiple individual silicone oils, the mixtures being adapted to the respective tasks and requirements that the oil must perform as a hydraulic fluid.

In other orthopedic devices, mineral oils or vegetable oils are used, which are also referred to as bio-oils. However, the field of application of bio-oils is limited as their viscosity is very dependent on temperature. They do, however, exhibit excellent lubrication properties and very effective wear and evaporation behavior.

The invention aims to improve the properties of the hydraulic fluid used and, in particular, to combine the only slight temperature dependence of the viscosity of the hydraulic fluid, which is achieved by the silicone oil, with good lubricating properties.

The invention solves the addressed task by way of an orthopedic device according to the preamble of claim 1, characterized in that the hydraulic fluid comprises a silicone oil and an additional oil which contains at least one ester group. In particular, an ester group has at least one double-bonded oxygen atom. To form the ester group, an acid group, for example a carboxy group (C—O—OH) is reacted with an alcohol to form an ester. Alternatively, a sulfonyl group, for example, can be reacted with an alcohol.

The invention is based on the surprising knowledge that a combination of silicone oil and an additive oil, which comprises at least one ester group, exhibits the desired properties and retains them in a hydraulic system of an orthopedic device. From the prior art it is known that silicone oil and such an additive oil, such as a bio-oil, cannot be permanently mixed. Even if an emulsion of both oils is made, the resulting hydraulic fluid separates after a while and the phases become separated.

Investigations have shown that this separation between the silicone oil and the additive oil also occurs within the hydraulic system of the orthopedic device, but surprisingly does not impede its function; rather, it supports it. Trials with orthopedic devices and the valves used in their hydraulic system have shown that the two oils can separate. In the process, the additive oil settles on the metallic surfaces located inside the hydraulic system, particularly inside the valve. This ensures optimum lubrication of the various components and a considerable reduction in friction. The silicone oil, on the other hand, deposits only slightly or even not at all on the metallic surfaces, but flows through the hydraulic system when it is stimulated and forced to do so by external forces. In this way, the excellent viscosity of the silicone oil, which can hardly be changed over a wide temperature range, can be used almost optimally.

While a large part of the additive oil consequently adheres to the surfaces inside the hydraulic system and is deposited and thus hardly moves when the hydraulic fluid flows, the main part of the silicone oil is responsible for the movement of the hydraulic fluid. The separation, which was initially considered a problem and deters the prior art from mixing other oils to a silicone oil, consequently has great advantages, which ensures that the two different oils can fully contribute their respective advantages and positive properties.

The double-bonded oxygen atoms of the ester groups and their strong electronegativity result in a negative partial charge on the oxygen atom. This leads to an increased electrical attraction to metals and plastics, which causes the additive oil to adhere to these surfaces. In addition, the partial electrical charges are likely to cause dipole-dipole interactions and probably the formation of hydrogen bonds, so that the additive oil forms a layer, preferably a laminar layer, on the surface of the components and thus displaces not only the silicone oil but also, in particular, water from these surfaces. This leads to the formation of a lubricating film on the surface of metallic components in particular.

The hydraulic fluid preferably comprises at most 25% additional oil, preferably at most 20% additional oil, especially preferably at most 15% additive oil. For example, the ratio of additive oil to the total amount of oil used is 2:26, 3:26 or 4:26, whereby the ratio of 3:26 has been proven to be particularly beneficial.

The additive oil preferably includes a glycol ester group and/or a triglyceride group. It is especially preferable if the additive oil is a bio-oil.

The invention also solves the task addressed by way of a method for filling a hydraulic system for an orthopedic device of the type described here, the method comprising the initial flushing of the hydraulic system with the additive oil and the subsequent filling with silicone oil.

Particularly preferably, during the flushing of the hydraulic system with additive oil, the hydraulic system is initially completely filled with additive oil and the additive oil is then drained again, wherein a proportion of additive oil remains in the hydraulic system. Particularly preferably, all of the additive oil or at least almost all of the additive oil, for example at least 90%, preferably at least 95% of the additive oil used, remains in the hydraulic system.

Preferably, at least some of the moving parts of the hydraulic system, especially preferably all moving parts of the hydraulic system, are wetted with additive oil before the hydraulic system is assembled. Alternatively, at least some of the moving parts, preferably all moving parts of the hydraulic system are wetted with additive oil after the hydraulic system has been assembled, but before it is filled with a hydraulic fluid.

The additive oil and the silicone oil are preferably filled into the hydraulic system at different positions. To this end, the hydraulic system has at least two different openings into which the hydraulic fluid can be filled. By using several different openings for the various components of the mixed hydraulic fluid, the two components can be introduced at the same time, without the mixture having to be produced beforehand.

The different oils are preferably mixed outside of the hydraulic system and then introduced into the hydraulic system. This can be done via multiple or a single filling point. Preferably, the silicone oil and the additive oil are mixed gravimetrically, i.e. by weight. The mix ratio specified here therefore preferably also applies for weight by percent.

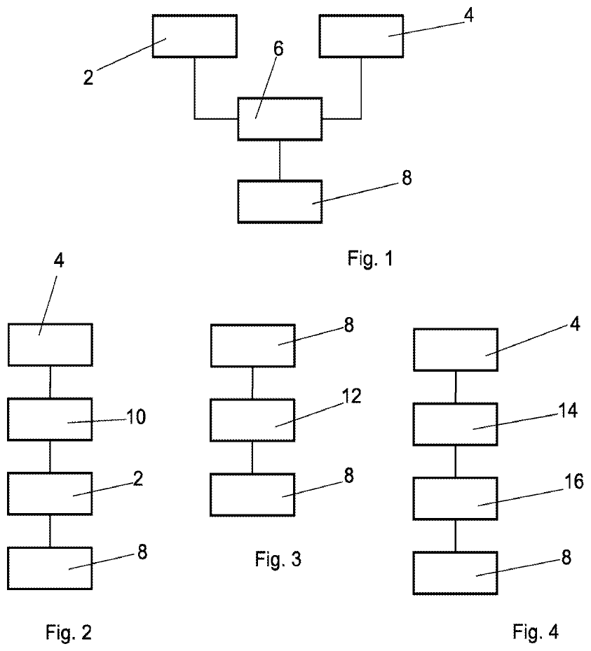

In the following, a number of embodiment examples of the invention will be explained in more detail with the aid of the accompanying figures. FIGS. 1 to 4 show different schematic methods for filling a hydraulic system.

The method according to FIG. 1 begins with the provision of the silicone oil 2 and of the additive oil 4. The silicone oil and the additive oil are mixed in a mixing step 6, the desired ratio of additive oil to silicone oil being set in the process. The hydraulic system is subsequently filled with the mixed oils.

FIG. 2 shows a different method. It begins with the provision of the additive oil 4, which is used to flush the hydraulic system in a flushing step 10. Additive oil introduced into the hydraulic system at a first filling point runs, if possible, through the entire hydraulic system and leaves the hydraulic system at a drainage point. If necessary, it is sufficient to flush only parts with the additive oil, for example the metallic components and/or the components that have a metallic surface which at least partially comes into contact with the hydraulic fluid. It is important to note in this method that in the flushing step 10, the additive oil is merely passed through the hydraulic system or a part of the hydraulic system. This is followed by the provision of the silicone oil 2 and the subsequent filling 8, the hydraulic system, which has already been flushed with the additive oil, being filled with the silicone oil during the filling 8. A mixing step 6 is unnecessary with this method. The mixture ratio between the additive oil and the silicone oil, which are inside the hydraulic system upon completion of the method according to FIG. 2, is determined according to the amount of additive oil that remains adhered to the surfaces within the hydraulic system in the flushing step 10 and therefore does not leave the hydraulic system in the flushing step 10. The individual surfaces of the part of the hydraulic system that is flushed with the additive oil in the flushing step 10 remain wetted with the additive oil, so that a certain amount of the additive oil remains within the hydraulic system. During filling 8, the hydraulic system is filled with silicone oil.

FIG. 3 shows a variation of the method. It does not include a flushing step 10; rather, the hydraulic system is completely filled with the additive oil upon initial filling 8. This is followed by an emptying 12, during which the additive oil introduced into the hydraulic system during initial filling 8 is drained off again. The second filling 8 then occurs, during which the hydraulic system emptied in this way is filled with silicone oil. As in the flushing step 10 according to FIG. 2, a certain amount of the additive oil remains within the hydraulic system upon the first filling 8 and subsequent emptying 12. The surfaces of the hydraulic system that come into contact with the hydraulic fluid remain wetted with the additive oil. The hydraulic system is then filled with silicone oil during the second filling 8.

FIG. 4 depicts a further embodiment example of the present invention. This method begins with the provision of the additive oil 4. During the subsequent wetting 14, at least some of the moving parts, preferably all moving parts, of the hydraulic system are wetted with the additive oil. This is followed by the assembly 16 of the hydraulic system, during which the wetted moving parts are installed. This also results in a hydraulic system whose surfaces that come into contact with the hydraulic fluid are wetted with additive oil. In the method according to FIG. 4, this is also followed by the filling 8 of the hydraulic system with the silicone oil.

### REFERENCE LIST

2 provision of the silicone oil
4 provision of the additive oil
6 mixing step
8 filling
10 flushing step
12 emptying
14 wetting
16 assembly

1. An orthopedic device, comprising at least one joint; and
a hydraulic system with at least one valve, the hydraulic system being filled with a hydraulic fluid,
wherein the hydraulic fluid comprises a silicone oil and an additive oil, wherein the additive oil contains at least one ester group.

2. The orthopedic device according to claim 1, wherein the hydraulic fluid comprises at most 25% additive oil.

3. The orthopedic device according to claim 1, wherein the additive oil contains a glycol ester group and/or a triglyceride group.

4. The orthopedic device according to claim 1, wherein the additive oil comprises at least one bio-oil.

5. A method for filling a hydraulic system for an orthopedic device according to claim 1, comprising
initially flushing the hydraulic system with additive oil; and then
filling the hydraulic system with silicone oil.

6. The method according to claim 5, wherein during the flushing of the hydraulic system with additive oil, the hydraulic system is initially completely filled with additive oil and the additive oil is then drained again, wherein a proportion of additive oil remains in the hydraulic system.

7. The method according to claim 5, wherein at least some moving parts of the hydraulic system are wetted with the additive oil prior to assembly of the hydraulic system.

8. The method according to claim 5, wherein the additive oil and the silicone oil are introduced into the hydraulic system at different points.

9. The method according to claim 5, wherein the additive oil and the silicone oil are mixed outside of the hydraulic system and then filled into the hydraulic system.

10. The orthopedic device according to claim 1, wherein the hydraulic fluid comprises at most 20% additive oil.

11. The orthopedic device according to claim 1, wherein the hydraulic fluid comprises at most 15% additive oil.

12. The method according to claim 7, wherein all moving parts of the hydraulic system are wetted with the additive oil prior to assembly of the hydraulic system.

13. The method according to claim 9, wherein the additive oil and the silicone oil are gravimetrically mixed outside of the hydraulic system and then filled into the hydraulic system.
